(19) United States
(12) Patent Application Publication
(10) Pub. No.: US 2024/0414135 A1
(43) Pub. Date: Dec. 12, 2024

(54) METHODS AND SYSTEMS OF TELEMEDICINE DIAGNOSTICS THROUGH REMOTE SENSING

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 18/807,509

(22) Filed: Aug. 16, 2024

Related U.S. Application Data
(63) Continuation-in-part of application No. 18/108,913, filed on Feb. 13, 2023, now Pat. No. 12,101,300.
Application No. 18/108,913 is a continuation of application No. 17/087,736, filed on Nov. 3, 2020, now Pat. No. 11,582,200.
Application No. 17/087,736 is a continuation of application No. 16/939,373, filed on Jul. 27, 2020, now Pat. No. 10,931,643.

(51) Int. Cl. H04L 9/40; A61B 5/00; G06F 21/62; G06Q 50/00; G16H 10/20; G16H 40/67; G16H 50/20; G16H 50/30; G16H 80/00; H04L 65/65

(52) U.S. Cl. CPC H04L 63/04; A61B 5/0022; A61B 5/7221; A61B 5/7267; A61B 5/7465; G16H 10/20; G16H 40/67; G16H 50/20; G16H 50/30; G16H 80/00; H04L 65/65; G06F 21/6245; G06Q 50/01; H04L 63/0428

(57) ABSTRACT

A system for telemedicine diagnostics through remote sensing may include at least a processor, and a memory communicatively connected to the at least processor, wherein the memory contains instructions configuring the at least processor to communicate to a subject a prompt, receive from the subject a response, classify the response to a medical condition category, generate a medical condition report as a function of the medical condition category, and initiate a telemedicine session by connecting the subject with a medical professional as a function of the response, and transmitting the medical condition report to a device operated by the medical professional.

### CROSS-REFERENCE TO RELATED APPLICATIONS

This application is a continuation-in-part of Nonprovisional application Ser. No. 18/108,913 filed on Feb. 13, 2023, and entitled “METHODS AND SYSTEMS OF TELEMEDICINE DIAGNOSTICS THROUGH REMOTE SENSING,” which is a continuation of Nonprovisional application Ser. No. 17/087,736 filed on Nov. 3, 2020, now U.S. Pat. No. 11,582,200, issued on Feb. 14, 2023, and entitled “METHODS AND SYSTEMS OF TELEMEDICINE DIAGNOSTICS THROUGH REMOTE SENSING,” which is a continuation of Nonprovisional application Ser. No. 16/939,373 filed on Jul. 27, 2020, now U.S. Pat. No. 10,931,643, issued on Feb. 23, 2021, and entitled “METHODS AND SYSTEMS OF TELEMEDICINE DIAGNOSTICS THROUGH REMOTE SENSING,” the entirety each of which is incorporated herein by reference.

### FIELD OF THE INVENTION

The present invention generally relates to the field of network communication. In particular, the present invention is directed to methods and systems of telemedicine diagnostics through remote sensing.

### BACKGROUND

Network connections can be susceptible to attack, leading to publication of private and sensitive information. Frequently, this can leave users unable to securely communicate, particularly in situations in need of immediate attention.

### SUMMARY OF THE DISCLOSURE

In an aspect, a system for telemedicine diagnostics through remote sensing may include at least a processor, and a memory communicatively connected to the at least processor, wherein the memory contains instructions configuring the at least processor to communicate to a subject a prompt, receive from the subject a response, classify the response to a medical condition category, generate a medical condition report as a function of the medical condition category, and initiate a telemedicine session by connecting the subject with a medical professional as a function of the response, and transmitting the medical condition report to a device operated by the medical professional.

In another aspect, a method of telemedicine diagnostics through remote sensing may include, using at least a processor, communicating to a subject a prompt, using the at least a processor, receiving from the subject a response, using the at least a processor, classifying the response to a medical condition category, using the at least a processor, generating a medical condition report as a function of the medical condition category, and using the at least a processor, initiating a telemedicine session by connecting the subject with a medical professional as a function of the response, and transmitting the medical condition report to a device operated by the medical professional.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein: FIG. 1 is a schematic diagram of an exemplary embodiment of a system for telemedicine diagnostics through remote sensing;

FIG. 2 is a block diagram of an exemplary embodiment of a system for telemedicine diagnostics through remote sensing;

FIG. 3 is a block diagram of an exemplary embodiment of a machine learning model;

FIG. 4 is a schematic diagram of an exemplary embodiment of a neural network;

FIG. 5 is a schematic diagram of an exemplary embodiment of a neural network node;

FIG. 6 is a block diagram of an exemplary embodiment of a subject database;

FIG. 7 is a diagram of an exemplary embodiment of a system for telemedicine diagnostics through remote sensing;

FIG. 8 is a diagram of an exemplary embodiment of a system including a chatbot;

FIG. 9 is a flow diagram of an exemplary embodiment of a method of telemedicine diagnostics through remote sensing;

FIG. 10 is a flow diagram depicting an exemplary embodiment of a method of telemedicine diagnostics through remote sensing;

FIG. 11 is a block diagram of a computing system that can be used to implement any one or more of the methodologies disclosed herein and any one or more portions thereof. The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

Embodiments disclosed herein use classification of remote sensor data to clinical measurements to determine probable clinical measurement results during telemedicine sessions. Follow-up remote sensor capture may be performed based on confidence levels or user inputs. Detection of need for clinical measurements may be performed as well.

Referring now to FIG. 1, an exemplary embodiment of a system for telemedicine diagnostics through remote sensing is illustrated. System includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently, or may include two or more computing device 104 operating in concert, in parallel, sequentially or the like; two or more computing devices may be included together in a single computing device 104 or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices in a first location and a second computing device 104 or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device 104, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device 104.

Computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 is configured to initiate a communication channel interface between the computing device 104 and a client device 112 operated by a human subject 116. A “human subject,” as used in this disclosure, is a person at a client device 112 receiving telemedicine services such as a virtual doctor's visit, physical, “checkup,” or the like. A “communication channel interface,” as used in this disclosure, is a communication medium within an interface. A communication channel interface may include an application, script, and/or program capable of providing a means of communication between at least two parties, including any oral and/or written forms of communication. A communication channel interface may allow computing device 104 to interface with electronic devices through graphical icons, audio indicators including primary notation, text-based user interfaces, typed command labels, text navigation, and the like. A communication channel interface may include slides or other commands that may allow a user 120 to select one or more options. A communication channel interface may include free form textual entries, where a user 120 may type in a response and/or message. A communication channel interface includes a display interface. Display interface includes a form or other graphical element having display fields, where one or more elements of information may be displayed. Display interface may display data output fields including text, images, or the like containing one or more messages. A communication channel interface may include data input fields such as text entry windows, drop-down lists, buttons, checkboxes, radio buttons, sliders, links, or any other data input interface that may capture user interaction as may occur to persons skilled in the art upon reviewing the entirety of this disclosure. A communication channel interface may be provided, without limitation, using a web browser, a native application, a mobile application, and the like.

With continued reference to FIG. 1, computing device 104 initiates a communication channel interface with a client device 112. A “client device,” as used in this disclosure, is a second computing device 104, including for example a mobile device such as a smartphone, tablet, laptop, desktop, and/or any other type of device suitable for use as computing device 104. Client device 112 is operated by a human subject 116; human subject 116 may include a person to whom telemedicine services are being rendered, including without limitation a patient. Computing device 104 may initiate communication channel interface using any network methodology as described herein. In an embodiment, a communication channel interface may be utilized to facilitate communications between a client device 112 operated by a human subject 116, and computing device 104 which may be operated by a user 120; user 120 may include a doctor, nurse, nurse practitioner, medical technician, medical assistant, pharmacist, pharmacy technician, and/or any other medical professional. For example, client device 112 may be operated by a patient who is in communication with a medical professional operating computing device 104, and communication channel interface may be utilized to have a telemedicine appointment. In yet another non-limiting example, client device 112 may be operated by a first member of a support group, and computing device 104 may be operated by a second member of the support group, whereby communication channel interface may be utilized to facilitate support group meetings and secure communications between members of the support group.

Further referring to FIG. 1, display interface may include a secure display interface, which may be implemented, maintained, and/or validated according to any process as described in U.S. Nonprovisional application Ser. No. 16/919,674, filed on Jul. 2, 2020, and entitled “METHODS AND SYSTEMS FOR GENERATING A SECURE COMMUNICATION CHANNEL INTERFACE FOR STREAMING OF SENSITIVE CONTENT,” the entirety of which is incorporated herein by reference.

With continued reference to FIG. 1, an as a non-limiting example, initiating a secure communication channel interface 108 may include transmitting to user client device 112 a configuration packet uniquely identifying computing device 104. A “configuration packet,” as used in this disclosure, is an encrypted message including a non-public device identifier.” An encrypted message includes any language that contains text, characters, and/or symbols that have been converted into an alternative form, such as but not limited to ciphertext. An encrypted message may include using an algorithm and/or a series of algorithms to transform plaintext messages into ciphertext. Encrypted messages may only be viewed in a non-encrypted from by decrypting it using a correct decryption key. Encrypted messages may be decrypted using both symmetric and asymmetric cryptographic key pairs, such as for example a public and private key pair. An encrypted message may be generated in a manner that complies with the Health Insurance Portability and Accountability Act (HIPPA) of 1996. A message may be encrypted using a pseudo-random encryption key generated by an algorithm. In one embodiment, a process of converting plaintext into ciphertext is known as “encryption.” Encryption process may involve the use of a datum, known as an “encryption key,” to alter plaintext. Cryptographic system may also convert ciphertext back into plaintext, which is a process known as “decryption.” Decryption process may involve the use of a datum, known as a “decryption key,” to return the ciphertext to its original plaintext form. In embodiments of cryptographic systems that are “symmetric,” decryption key is essentially the same as encryption key: possession of either key makes it possible to deduce the other key quickly without further secret knowledge. Encryption and decryption keys in symmetric cryptographic systems may be kept secret and shared only with persons or entities that the user of the cryptographic system wishes to be able to decrypt the ciphertext. One example of a symmetric cryptographic system is the Advanced Encryption Standard (“AES”), which arranges plaintext into matrices and then modifies the matrices through repeated permutations and arithmetic operations with an encryption key.

With continued reference to FIG. 1, a “non-public device identifier,” as used in this disclosure, is a decryption key that cannot be readily deduced without additional secret knowledge, such as for example, a private key. A non-public device identifier may include a randomly generated number that cannot be easily guessed. A non-public device identifier may be generated using a stream cipher and/or a block cipher. An encrypted message may be transmitted with a non-public device identifier, to initiate secure communication between computing device 104 and user client device 112.

With continued reference to FIG. 1, computing device 104 receives from user device 104 a confirmation authentication a configuration packet. A confirmation may include any message, that allows user client device 112 to confirm the identify and/or authenticity of computing device 104. A confirmation may be transmitted from user client device 112 to computing device 104 using any network methodology as described herein. In an embodiment, a confirmation authentication may include receiving from user client device 112 a configuration packet uniquely identifying user client device 112. In such an instance, computing device 104 may receive the configuration packet uniquely identifying user client device 112 and authenticate the configuration packet, and the identify of user client device 112. Computing device 104 establishes a communication exchange as a function of receiving from user client device 112, a confirmation authenticating the configuration packet. A communication exchange includes any telecommunication handshake that includes an automated process of communications between two or more participants, such as computing device 104 and user client device 112. A telecommunication handshake includes the exchange of information establishing protocols of communication at the start of communication before full communication commences. A telecommunication handshake may include exchanging signals to establish a communication link as well as to agree as to which protocols to implement. A telecommunication handshake May include negotiating parameters to be utilized between user client device 112 and computing device 104, including information transfer rate, coding alphabet, parity, interrupt procedure, and/or any other protocol or hardware features. A telecommunication handshake may include but is not limited to a transmission control protocol (TCP), simple mail transfer protocol (SMTP), transport layer security (TLS), Wi-Fi protected access (WPA), and the like.

With continued reference to FIG. 1, a communication channel interface includes an audiovisual capture device 124. An “audiovisual capture device,” as used in this disclosure, is a device used to record sound and/or images. An audiovisual capture device 124 may include but is not limited to, a camera, a video camera, a mobile device, a recording device, a DVD player, a sensor, a television tuner, a video capture card, a universal serial bus (USB) audio and/or visual capture device, and the like. In an embodiment, an audiovisual capture device 124 may be located within client device 112.

Still referring to FIG. 1, communication interface includes an audiovisual streaming protocol. An “audiovisual streaming protocol,” as used in this disclosure, is a packet-based communication protocol that streams video and/or audio data from one device to another and vice-versa. An audiovisual streaming protocol may support a “video chat” process whereby a user 120 of computer device can see real-time or near real-time footage of human subject 116, while human subject 116 may be able to see real-time or near real-time footage of user of computing device 104. User 120 of computing device 104 may include, without limitation, a doctor, physician, nurse practitioner, nurse, therapist, psychologist, medical technician, and/or any other medical professional and/or assistant thereof. Audiovisual streaming protocol may enable user to perform many actions of a medical visit virtually, for instance by having human subject 116 perform measurements of height and/or weight of human subject 116, by having human subject 116 present different body parts for inspection using audiovisual capture device 124, or the like.

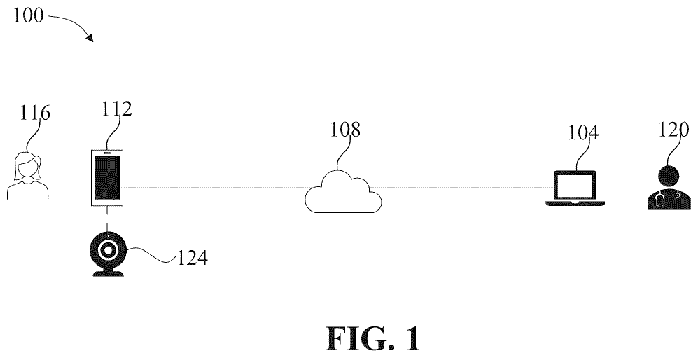

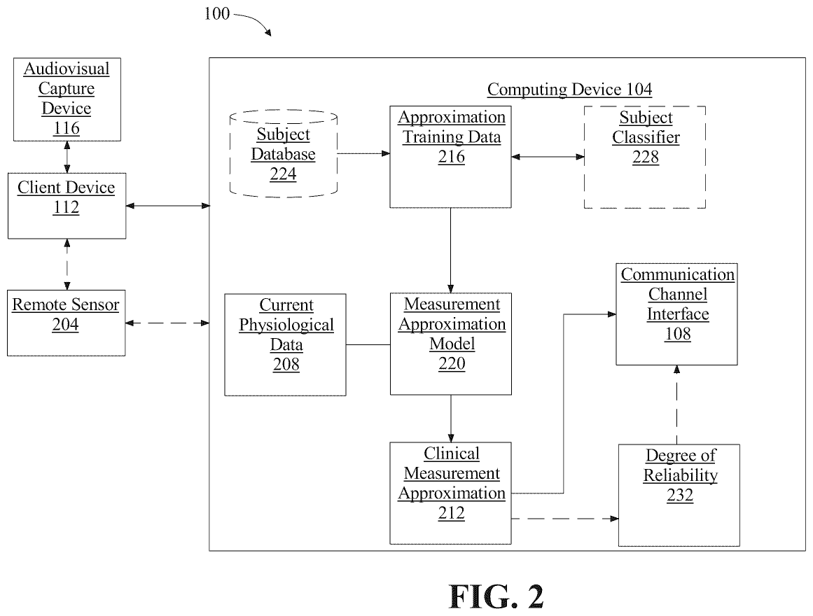

Referring now to FIG. 2, computing device 104 is configured to receive a plurality of current physiological data 208 from at least a remote sensor 204 at the human subject 116. A “remote sensor,” as used in this disclosure, is a device that captures data of human subject 116 and transmits that data to computing device 104, either by transmitting the data to client device 112 which relays the data to computing device 104, or by transmitting the data separately over a network connection. Data may be transmitted via communication channel interface and/or via a separate network connection formed, for instance, using a secure sockets layer (SSL) and/or hypertext transfer protocol-secure (HTTPS) process. Remote sensor 204 may include, without limitation, a camera such as a digital camera incorporated in a mobile device or the like, a microphone such as a mobile device microphone, a motion sensor, which may include one or more accelerometers, gyroscopes, magnetometer, or the like. Remote sensor 204 may include one or more peripheral devices such as a peripheral pulse oximeter or the like. Remote sensor 204 may include a network-connected device such as a network connected digital scale or the like. In an embodiment, remote sensor 204 may be used to capture audio or visual data concerning one or more portions of human subject 116's anatomy. For instance, and without limitation, a microphone may be pressed against one or more portions of human subject 116 at direction of user 120 over communication channel, causing capture of audio data from the one or more portion of human subject 116; as a non-limiting example, audio data of human subject 116 lungs, heart, digestive system, or the like may be so captured. As a further example, user 120 may instruct human subject 116 to train a camera on one or more portions of anatomy to capture visual data concerning such one or more portions. Such physiological data may be combined; for instance, audio capture of circulatory system noise data may be combined with pulse oximetry data from a peripheral pulse oximeter and/or motion-sensor data indicating a degree of activity. Remote sensor 204 may include an electrical sensor such as a portable electrocardiogram device or the like. Generally, any sensor capable of capturing data of human subject 116 and transmitting such data locally or over a network may be used as a remote sensor 204.

Still referring to FIG. 2, plurality of current physiological data 208 may include cardiovascular data such as heart rate data, blood pressure data, or the like, for instance captured using audio and/or oximetry devices. Plurality of current physiological data 208 may include respiratory data such as audio capture of pulmonary sounds using a microphone or the like. Plurality of current physiological data 208 may include neurological data. Plurality of current physiological data 208 may include digestive audio data. Plurality of physiological data may include visual data captured regarding one or more elements of externally visible patient anatomy. Plurality of physiological data may capture one or more elements of human subject 116 bodily motion, including gait, posture, or gestural motions.

Still referring to FIG. 2, computing device 104 is configured to generate a clinical measurement approximation 212 as a function of the plurality of current physiological data 208. A “clinical measurement approximation,” as used in this disclosure, is a numerical value estimating a likely clinical measurement matching plurality of current physiological data 208. A clinical measurement approximation 212 may function as approximation of what a doctor would get in person. For instance, a clinical measurement approximation 212 may approximate a heart rate, blood pressure, oxygen level, or other “vital sign” that might be captured in a clinical setting. In an embodiment, remote sensor 204 data may lack accuracy of clinically measured data and/or may not measure a given clinically measured datum directly. For instance, heart rate and/or blood oxygen as measured by home equipment, mobile devices, and/or mobile device peripherals may be less accurate than similar measurements captured using professional equipment. As a further non-limiting example, remote sensors 204 available at human subject 116 and/or client device 112 may not measure blood pressure directly but may measure a combination of cardiovascular parameters having some correlation, singly or in combination, with blood pressure, which may be used in some combination to estimate blood pressure levels. Persons skilled in the art, upon reviewing the entirety of this disclosure, will be aware of various clinical measurement approximations 212 that may be generated. A user 120 of computing device 104 may use one or more clinical measurement approximations 212, in combination with other information obtained by communicating with human subject 116 and/or clinical history of human subject 116, to arrive at conclusions concerning a state of health of human subject 116.

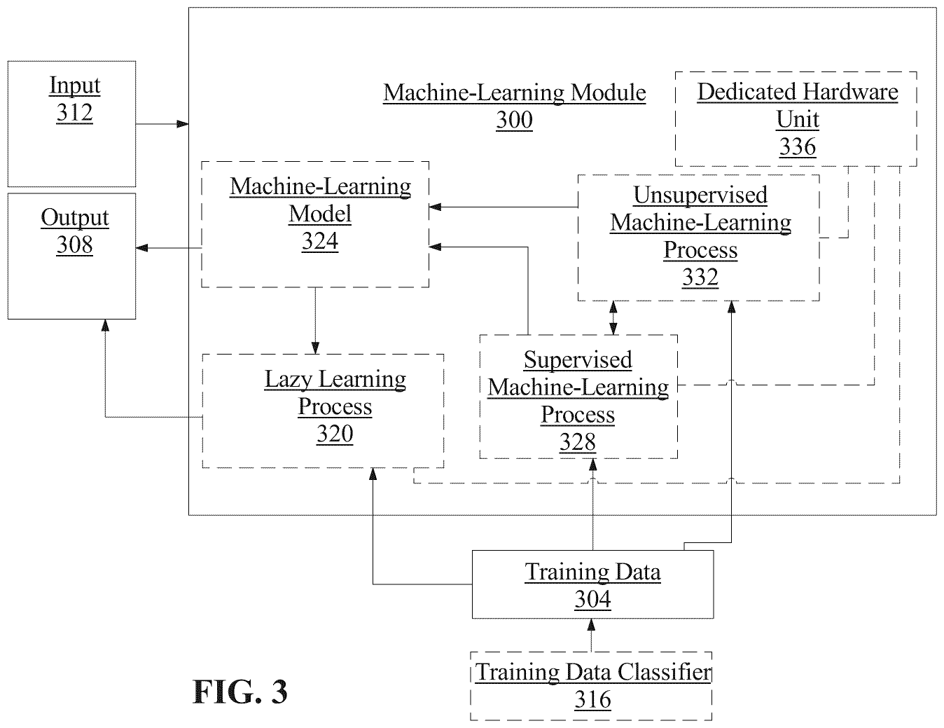

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to a cohort of persons and/or clinical data having similarities to data of human subject 116 and/or current physiological data 208.

Still referring to FIG. 3, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A)P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 3, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 3, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 3, computing device, processor, and/or module may down-sample elements of a training example to a desired lower number of data elements. As a non-limiting example, a high pixel count image may have 256pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 3, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 3, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{median}}{IQR} .$$

Persons skilled in the art, upon reviewing the entirety of this disclosure, will be aware of various alternative or additional approaches that may be used for feature scaling.

Further referring to FIG. 3, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described in this disclosure as described above as inputs, outputs as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 332 may not require a response variable; unsupervised processes 332 may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

With continued reference to FIG. 3, a system may use user feedback to train the machine-learning models and/or classifiers described above. For example, classifier may be trained using past inputs and outputs of classifier. In some embodiments, if user feedback indicates that an output of classifier was “bad,” then that output and the corresponding input may be removed from training data used to train classifier, and/or may be replaced with a value entered by, e.g., another user that represents an ideal output given the input the classifier originally received, permitting use in retraining, and adding to training data; in either case, classifier may be retrained with modified training data as described in further detail below. In some embodiments, training data of classifier may include user feedback.

With continued reference to FIG. 3, in some embodiments, an accuracy score may be calculated for classifier using user feedback. For the purposes of this disclosure, “accuracy score,” is a numerical value concerning the accuracy of a machine-learning model. For example, a plurality of user feedback scores may be averaged to determine an accuracy score. In some embodiments, a cohort accuracy score may be determined for particular cohorts of persons. For example, user feedback for users belonging to a particular cohort of persons may be averaged together to determine the cohort accuracy score for that particular cohort of persons and used as described above. Accuracy score or another score as described above may indicate a degree of retraining needed for a machine-learning model such as a classifier; a system may perform a larger number of retraining cycles for a higher number (or lower number, depending on a numerical interpretation used), and/or may collect more training data for such retraining, perform more training cycles, apply a more stringent convergence test such as a test requiring a lower mean squared error, and/or indicate to a user and/or operator that additional training data is needed.

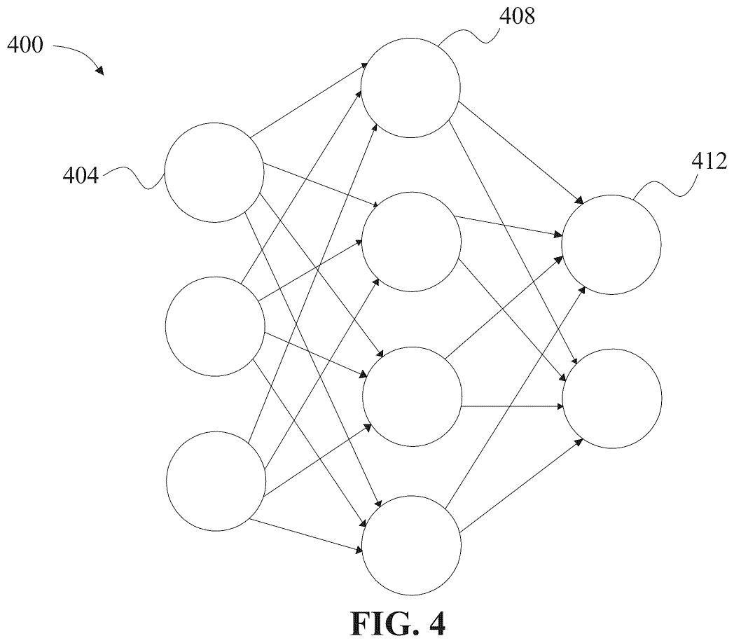

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes.

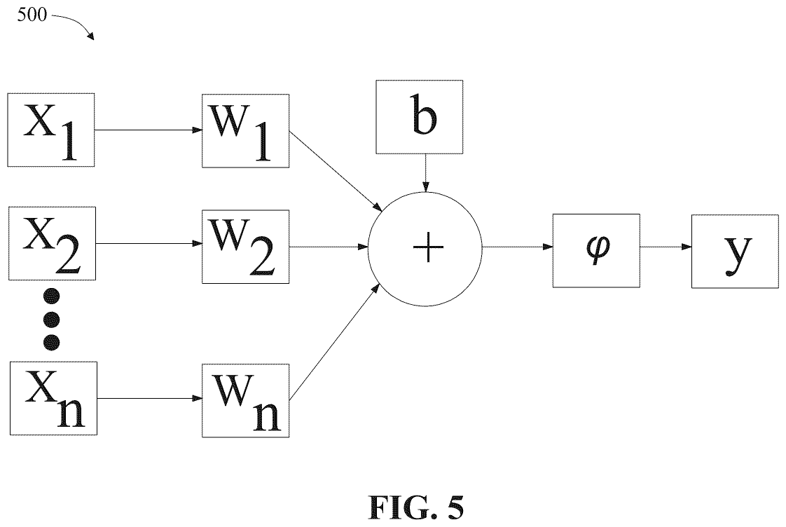

Referring now to FIG. 5, an exemplary embodiment of a node 500 of a neural network is illustrated. A node may include, without limitation a plurality of inputs xi that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0,x), a “leaky” and/or “parametric” rectified linear unit function such as f(x)=max(ax,x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Still referring to FIG. 5, a “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like. CNN may include, without limitation, a deep neural network (DNN) extension, where a DNN is defined as a neural network with two or more hidden layers.

Referring again to FIG. 2, and as a non-limiting example, computing device 104 may be configured to generate clinical measurement approximation 212 by receiving approximation training data 216 correlating physiological data with clinical measurement data, training a measurement approximation model 220 as a function of the training data and a machine-learning process, and generating the clinical measurement approximation 212 as a function of the current physiological data 208 and the measurement approximation model 220. “Clinical measurement data,” as used in this disclosure is data describing measurements taken in a clinical setting such as a doctor's office with clinical equipment, such as a blood pressure cuff, stethoscope, or the like. Approximation training data 216 may be collected, without limitation, by taking simultaneous clinical measurements and remote sensor 204 measurements of human subject 116 and/or other persons over one or more in-person clinical visits, for instance and without limitation according to examples as described in further detail below.

Further referring to FIG. 2, generating clinical measurement approximation 212 may include identifying at least a category of current physiological data 208 and classifying the at least a category of current physical data to the measurement approximation model 220 and/or to approximation training data 216 used to train approximation model. Classification may be performed using a classifier, as described above, which may match physiological data sets to one or more input sets suitable for one or more clinical measurement approximation 212. For instance, and without limitation, current physiological data 208 may contain a first set of data that may be used to approximate a first clinical measurement, and a second set of data that may be used to approximate a second clinical measurement; in this case, classification may identify both such clinical measurements and machine-learning models and/or training data that may be used to approximate each. Where the same clinical measurement may be approximated by a first set of physiological measurement data or a second set of physiological measurement data, classifier may identify both, and computing device 104 may determine which set of physiological measurement data produces a more reliable result, for instance as calculated below; a measurement approximation model 220 associated with the more reliable set may be selected for use by computing device 104.

Still referring to FIG. 2, training measurement approximation model 220 may include generating a general model. General model may be a first measurement approximation model 220 trained with general approximation training data 216, as described above, concerning multiple persons, where each entry may be obtained as described above. A population of persons that general approximation training data 216 describes may be a randomly selected population of patients and/or a population of patients chosen according to one or more characteristics shared with human subject 116, where one or more characteristics may include demographic data such as age, sex, ethnicity, region of residence, or the like, diagnostic data such as shared illnesses, conditions, and/or preconditions, other data such as user habits, or the like. Such data may be tracked and stored for each patient in a subject database 224.

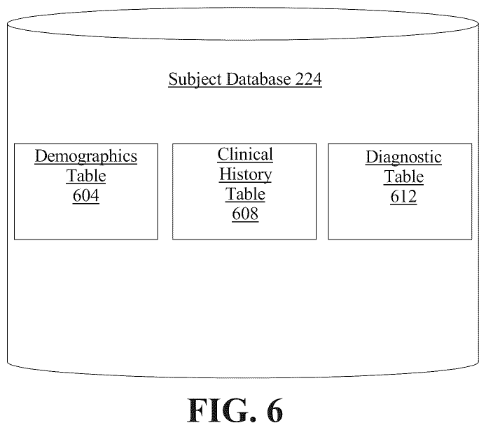

Referring now to FIG. 6, an exemplary embodiment of a subject database 224 is illustrated. Subject database may be implemented, without limitation, as a relational subject database, a key-value retrieval subject database such as a NOSQL subject database, or any other format or structure for use as a subject database that a person skilled in the art would recognize as suitable upon review of the entirety of this disclosure. Subject database may alternatively or additionally be implemented using a distributed data storage protocol and/or data structure, such as a distributed hash table or the like. Subject database may include a plurality of data entries and/or records as described above. Data entries in a subject database may be flagged with or linked to one or more additional elements of information, which may be reflected in data entry cells and/or in linked tables such as tables related by one or more indices in a relational subject database. Persons skilled in the art, upon reviewing the entirety of this disclosure, will be aware of various ways in which data entries in a subject database may store, retrieve, organize, and/or reflect data and/or records as used herein, as well as categories and/or populations of data consistently with this disclosure. Subject database 224 may include one or more tables, including without limitation a demographics table 604; demographics table may include demographic information concerning human subject 116, including without limitation age, ethnicity, location of residence, national origin, sex, or the like. Subject database 224 may include a clinical history table 608, which may contain previously captured clinical measurements of human subject 116 and/or related sensor measurements. Subject database 224 may include a diagnostic table 612, which may contain one or more diagnoses, prognoses, and/or other prognostic data concerning human subject 116.

Referring again to FIG. 2, approximation training data 216 and/or general approximation training data 216 may alternatively or additionally be matched to human subject 116 using a subject classifier 228. Subject classifier 228, which may include any classifier as described above, may classify approximation training data 216 to one or more patient categories, and may then classify human subject 116 to one or more such patient categories; corresponding training data may be used to train a general approximation model, which training may be performed after classification of human subject 116 and/or concurrently or prior to such classification. In an embodiment, a general approximation model may enable computing device 104 to approximate a given clinical measurement for persons in given cohort and/or for persons generally, providing a “first guess” regarding human subject 116, which may be used for further steps of process and/or used as an initial step in a human subject 116--specific training process. Still referring to FIG. 2, computing device 104 may further train general

approximation model using subject-specific training data, defined herein as approximation training data 216 collected using clinical measurements and corresponding remote sensor 204 data regarding human subject 116 specifically. This data may be collected in one or more live clinical visits. In an embodiment, training a subject-specific model from a general approximation model may enable a relatively sparse set of subject-specific training data to be used to generate an accurate subject-specific model.

In an embodiment, and with further reference to FIG. 2, training measurement approximation model 220 may include classification of the human subject 116 to approximation training data 216; for instance in lieu of use of use of subject-specific training data, training data classified to human subject as above may be used to generate approximation model.

In an embodiment, and still referring to FIG. 2, generating clinical measurement approximation 212 may include calculating a change between a first discrete data set of plurality of current physiological data 208 and a second discrete set of current physiological data 208 and generating clinical measurement approximation 212 as a function of the change. A “discrete set” of data, as used in this disclosure, is a set of data that may be definitely separated from another discrete set, such as data separated by an interval in time, taken with a different category of remote sensor 204, or the like. A difference between first discrete set and second discrete set may be used in some embodiments as an input to approximation model. In other words, some embodiments of system may use a change in sensor feedback from a first discrete measurement to a second discrete measurement captured after the first discrete measurement, and/or captured through a distinct channel, to generate clinical measurement approximation 212. First discrete set of current physiological data 208 may be temporally separated from second discrete set of current physiological data 208, where “temporal separation” indicates that a period of time, which may be a time in seconds, minutes, hours, days, or the like, separates the two sets. Period of time may be determined by a user 120 of computing device 104, such as a doctor or other medical professional.

Still referring to FIG. 2, computing device 104 may be configured to record the first discrete set of current physiological data 208, generate a prompt instructing the human subject 116 to perform an activity, and record the second discrete set of current physiological data 208. For instance, and without limitation, where the clinical measurement to be approximated is a cardiovascular measurement, activity may include some degree of physical exertion such as squats, running in place, walking around the room, bending down to touch toes, or the like. As a further example, activity may include consuming a food, liquid, or other substance, positioning a sensor by moving from a first position used to capture first data set to a second position used to capture second data set (e.g., motion from front of torso to rear of torso of a microphone), or the like. Computing device 104 may be configured to verify that the human subject 116 has performed the activity. Verification may be performed, without limitation, by way of visual confirmation by a user 120 of computing device 104, such as without limitation a doctor or other medical professional.

Still referring to FIG. 2, computing device 104 is configured to present clinical measurement approximation 212 to a user 120 of computing device 104 using the communication interface. User 120 of computing device 104 may include, without limitation, a doctor or other medical professional. Computing device 104 may be configured to determine a degree of reliability 232 of the clinical measurement and provide the degree of reliability 232 using the communication interface. A “degree of reliability,” as used in this disclosure, is a probability that clinical measurement approximation 212 matches a corresponding clinical measurement. Degree of reliability 232 may be computed, without limitation, using a terminal error function result in generation of approximation model; for instance, where error function was iteratively minimized during training, minimal error function resulting from training process may be converted to an error and/or error probability percentage. Computing device 104 may be configured to identify a follow-up action as a function of the degree of reliability 232. For instance, and without limitation, computing device 104 may compare degree of reliability 232 to a preconfigured threshold number, such as a probability of accuracy above a threshold percentage; falling below the threshold number may cause computing device 104 to determine that more information is needed. Alternatively or additionally, a user 120 of computing device 104 such as a doctor or other medical professional may view degree of reliability 232 as displayed to the user 120, may conclude that further information is needed for a more accurate result, and may enter an instruction via computing device 104 and/or interface indicating that a follow-up action is needed.

Further referring to FIG. 2, follow-up action may include one or more additional readings using remote sensors 204, which may include duplicate readings, readings taking using a different sensor and/or set of sensors, readings temporally separated from a first set of readings, for which changes as described above may be used as an input, or the like. A new set of remote sensor 204 readings may be combined with an original set as inputs to approximation model and/or may be input to approximation model separately; in either case, reliability of second output may be evaluated for improvement in reliability, and process may be repeated until reliability exceeds a threshold number as described above and/or a user 120 of computing device 104 enters a command indicating satisfaction. Follow-up action may alternatively or additionally include a clinical test corresponding to clinical measurement approximation 212. For instance, computing device 104 may output a suggestion to perform clinical test and/or a user 120 of computing device 104 may instruct human subject 116 to perform the clinical test and/or may order that a clinical test be performed. In an embodiment, processes described in this disclosure may enable a medical professional to determine when processes such as diagnostics using shipped samples, conveyance of more exact testing equipment to a location of human subject 116, and/or an in-person clinical visit are necessary or recommended.

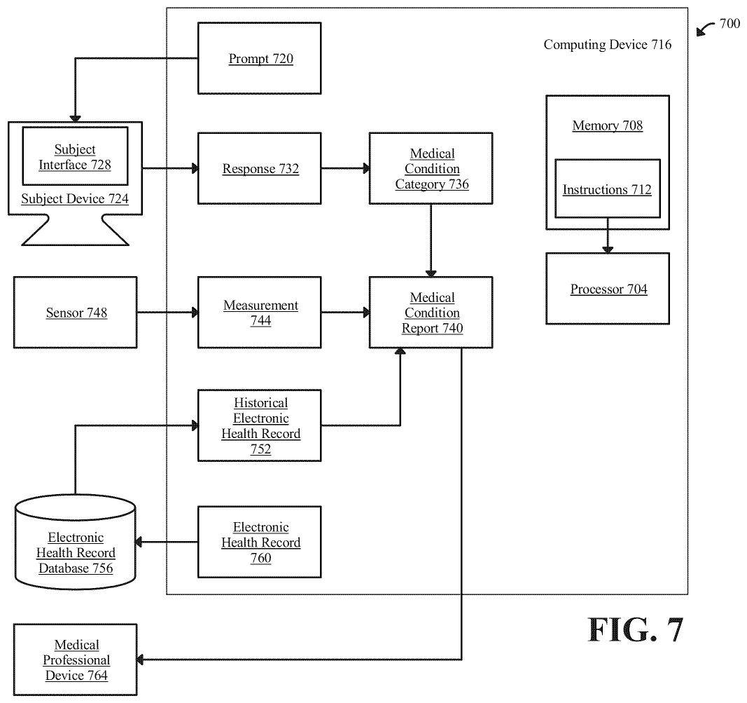

Referring now to FIG. 7, an exemplary embodiment of a system 700 for telemedicine diagnostics through remote sensing is illustrated. System 700 may include at least a processor 704 and a memory 708 communicatively connected to the at least a processor 704, the memory 708 containing instructions 712 configuring the at least a processor 704 to perform one or more processes described herein. Computing device 716 may include processor 704 and/or memory 708.

Still referring to FIG. 7, in some embodiments, system 700 may generate prompt 720. In some embodiments, system 700 may generate prompt 720 based on one or more default prompts. In some embodiments, system 700 may generate prompt 720 based on a historical electronic health record of a subject. In some embodiments, system 700 may generate prompt 720 based on a response by a subject to a previous prompt.

Still referring to FIG. 7, in some embodiments, system 700 may generate prompt 720 using a machine learning model such as a prompt generation machine learning model. Prompt generation machine learning model may use a machine learning algorithm described herein, such as a supervised learning algorithm, an unsupervised learning algorithm, a reinforcement learning algorithm, and the like. In some embodiments, prompt generation machine learning model may include a neural network model. In some embodiments, prompt generation machine learning model may include a language model. In some embodiments, a supervised learning algorithm and a training dataset are used to train prompt generation machine learning model; such training dataset may include example electronic health records and/or example responses to prior prompts, associated with example prompts. Such training data may be gathered by, for example, reviewing historical records of subject-medical professional interactions, identifying prompts and/or questions asked by medical professionals, and identifying a context of such prompts and/or questions. Once prompt generation machine learning model has been trained, it may be used to generate prompt 720. This may be done by inputting discussion a historical electronic health record and/or a response to a prior prompt into prompt generation machine learning model and receiving, as an output, prompt 720.

System 700 may transmit prompt 720 to subject device 724. Subject device 724 may display to a subject, by subject interface 728, prompt 720. Subject device 724 may include, in non-limiting examples, a smartphone, smartwatch, laptop computer, desktop computer, virtual reality device, or tablet. Subject device 724 may include an input interface and/or an output interface. An input interface may include one or more mechanisms for a computing device to receive data from a subject such as, in non-limiting examples, a mouse, keyboard, button, scroll wheel, camera, microphone, switch, lever, touchscreen, trackpad, joystick, and controller. An output interface may include one or more mechanisms for a computing device to output data to a user such as, in non-limiting examples, a screen, speaker, and haptic feedback system. An output interface may be used to display one or more elements of data described herein. As used herein, a device “displays” a datum if the device outputs the datum in a format suitable for communication to a user. For example, a device may display a datum by outputting text or an image on a screen or outputting a sound using a speaker.

Still referring to FIG. 7, in some embodiments, system 700 may receive response 732. As used herein, a “response” is a datum describing an input by a user, a lack of an input by a user, or both, where the input, lack of input, or both occurs after a prompt and is based on the prompt. Response 732 may include a datum describing, in non-limiting examples, an audio file including subject speech which is responsive to prompt 720, a text file including text input by a subject where the text is responsive to prompt 720, and an image uploaded by subject where the image includes information relevant to prompt 720.

Still referring to FIG. 7, in some embodiments, response 732 may include audio data, and such audio data may be transcribed and/or processed using an automatic speech recognition process. In some embodiments, automatic speech recognition may require training (i.e., enrollment). In some cases, training an automatic speech recognition model may require an individual speaker to read text or isolated vocabulary. In some cases, training data may include an audio component having an audible verbal content, the contents of which are known a priori by a computing device. Computing device may then train an automatic speech recognition model according to training data which includes audible verbal content correlated to known content. In this way, computing device may analyze a person's specific voice and train an automatic speech recognition model to the person's speech, resulting in increased accuracy. Alternatively, or additionally, in some cases, computing device may include an automatic speech recognition model that is speaker independent. As used in this disclosure, a “speaker independent” automatic speech recognition process is an automatic speech recognition process that does not require training for each individual speaker. Conversely, as used in this disclosure, automatic speech recognition processes that employ individual speaker specific training are “speaker dependent.”

Still referring to FIG. 7, in some embodiments, an automatic speech recognition process may perform voice recognition or speaker identification. As used in this disclosure, “voice recognition” is a process of identifying a speaker, from audio content, rather than what the speaker is saying. In some cases, computing device may first recognize a speaker of verbal audio content and then automatically recognize speech of the speaker, for example by way of a speaker dependent automatic speech recognition model or process. In some embodiments, an automatic speech recognition process can be used to authenticate or verify an identity of a speaker. In some cases, a speaker may or may not include subject. For example, subject may speak within response 732, but others may speak as well.

Still referring to FIG. 7, in some embodiments, an automatic speech recognition process may include one or all of acoustic modeling, language modeling, and statistically based speech recognition algorithms. In some cases, an automatic speech recognition process may employ hidden Markov models (HMMs). As discussed in greater detail below, language modeling such as that employed in natural language processing applications like document classification or statistical machine translation, may also be employed by an automatic speech recognition process.

Still referring to FIG. 7, an exemplary algorithm employed in automatic speech recognition may include or even be based upon hidden Markov models. Hidden Markov models (HMMs) may include statistical models that output a sequence of symbols or quantities. HMMs can be used in speech recognition because a speech signal can be viewed as a piecewise stationary signal or a short-time stationary signal. For example, over a short time scale (e.g., 10milliseconds), speech can be approximated as a stationary process. Speech (i.e., audible verbal content) can be understood as a Markov model for many stochastic purposes.

Still referring to FIG. 7, in some embodiments HMMs can be trained automatically and may be relatively simple and computationally feasible to use. In an exemplary automatic speech recognition process, a hidden Markov model may output a sequence of n-dimensional real-valued vectors (with n being a small integer, such as 10), at a rate of about one vector every 10 milliseconds. Vectors may consist of cepstral coefficients. A cepstral coefficient requires using a spectral domain. Cepstral coefficients may be obtained by taking a Fourier transform of a short time window of speech yielding a spectrum, decorrelating the spectrum using a cosine transform, and taking first (i.e., most significant) coefficients. In some cases, an HMM may have in each state a statistical distribution that is a mixture of diagonal covariance Gaussians, yielding a likelihood for each observed vector. In some cases, each word, or phoneme, may have a different output distribution; an HMM for a sequence of words or phonemes may be made by concatenating an HMMs for separate words and phonemes.

Still referring to FIG. 7, in some embodiments, an automatic speech recognition process may use various combinations of a number of techniques in order to improve results. In some cases, a large-vocabulary automatic speech recognition process may include context dependency for phonemes. For example, in some cases, phonemes with different left and right context may have different realizations as HMM states. In some cases, an automatic speech recognition process may use cepstral normalization to normalize for different speakers and recording conditions. In some cases, an automatic speech recognition process may use vocal tract length normalization (VTLN) for male-female normalization and maximum likelihood linear regression (MLLR) for more general speaker adaptation. In some cases, an automatic speech recognition process may determine so-called delta and delta-delta coefficients to capture speech dynamics and might use heteroscedastic linear discriminant analysis (HLDA). In some cases, an automatic speech recognition process may use splicing and a linear discriminate analysis (LDA)-based projection, which may include heteroscedastic linear discriminant analysis or a global semi-tied covariance transform (also known as maximum likelihood linear transform [MLLT]). In some cases, an automatic speech recognition process may use discriminative training techniques, which may dispense with a purely statistical approach to HMM parameter estimation and instead optimize some classification-related measure of training data; examples may include maximum mutual information (MMI), minimum classification error (MCE), and minimum phone error (MPE).

Still referring to FIG. 7, in some embodiments, an automatic speech recognition process may be said to decode speech (i.e., audible verbal content). Decoding of speech may occur when an automatic speech recognition system is presented with a new utterance and must compute a most likely sentence. In some cases, speech decoding may include a Viterbi algorithm. A Viterbi algorithm may include a dynamic programming algorithm for obtaining a maximum a posteriori probability estimate of a most likely sequence of hidden states (i.e., Viterbi path) that results in a sequence of observed events. Viterbi algorithms may be employed in context of Markov information sources and hidden Markov models. A Viterbi algorithm may be used to find a best path, for example using a dynamically created combination hidden Markov model, having both acoustic and language model information, using a statically created combination hidden Markov model (e.g., finite state transducer [FST] approach).

Still referring to FIG. 7, in some embodiments, speech (i.e., audible verbal content) decoding may include considering a set of good candidates and not only a best candidate, when presented with a new utterance. In some cases, a better scoring function (i.e., re-scoring) may be used to rate each of a set of good candidates, allowing selection of a best candidate according to this refined score. In some cases, a set of candidates can be kept either as a list (i.e., N-best list approach) or as a subset of models (i.e., a lattice). In some cases, re-scoring may be performed by optimizing Bayes risk (or an approximation thereof). In some cases, re-scoring may include optimizing for sentence (including keywords) that minimizes an expectancy of a given loss function with regards to all possible transcriptions. For example, re-scoring may allow selection of a sentence that minimizes an average distance to other possible sentences weighted by their estimated probability. In some cases, an employed loss function may include Levenshtein distance, although different distance calculations may be performed, for instance for specific tasks. In some cases, a set of candidates may be pruned to maintain tractability.

Still referring to FIG. 7, in some embodiments, an automatic speech recognition process may employ dynamic time warping (DTW)-based approaches. Dynamic time warping may include algorithms for measuring similarity between two sequences, which may vary in time or speed. For instance, similarities in walking patterns would be detected, even if in one video the person was walking slowly and if in another he or she were walking more quickly, or even if there were accelerations and deceleration during the course of one observation. DTW has been applied to video, audio, and graphics-indeed, any data that can be turned into a linear representation can be analyzed with DTW. In some cases, DTW may be used by an automatic speech recognition process to cope with different speaking (i.e., audible verbal content) speeds. In some cases, DTW may allow computing device to find an optimal match between two given sequences (e.g., time series) with certain restrictions. That is, in some cases, sequences can be “warped” non-linearly to match each other. In some cases, a DTW-based sequence alignment method may be used in context of hidden Markov models.

Still referring to FIG. 7, in some embodiments, an automatic speech recognition process may include a neural network. Neural network may include any neural network, for example those disclosed with reference to FIGS. 3-5. In some cases, neural networks may be used for automatic speech recognition, including phoneme classification, phoneme classification through multi-objective evolutionary algorithms, isolated word recognition, audiovisual speech recognition, audiovisual speaker recognition and speaker adaptation. In some cases, neural networks employed in automatic speech recognition may make fewer explicit assumptions about feature statistical properties than HMMs and therefore may have several qualities making them attractive recognition models for speech recognition. When used to estimate the probabilities of a speech feature segment, neural networks may allow discriminative training in a natural and efficient manner. In some cases, neural networks may be used to effectively classify audible verbal content over short-time interval, for instance such as individual phonemes and isolated words. In some embodiments, a neural network may be employed by automatic speech recognition processes for pre-processing, feature transformation and/or dimensionality reduction, for example prior to HMM-based recognition. In some embodiments, long short-term memory (LSTM) and related recurrent neural networks (RNNs) and Time Delay Neural Networks (TDNN's) may be used for automatic speech recognition, for example over longer time intervals for continuous speech recognition.

Still referring to FIG. 7, in some embodiments, a language model may be used to process response 732. As used herein, a “language model” is a program capable of interpreting natural language, generating natural language, or both. In some embodiments, a language model may be configured to interpret the output of an automatic speech recognition function and/or an OCR function. A language model may include a neural network. A language model may be trained using a dataset that includes natural language.

Still referring to FIG. 7, in some embodiments, a language model may be configured to extract one or more words from a document. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols. Textual data may be parsed into tokens, which may include a simple word (sequence of letters separated by whitespace) or more generally a sequence of characters. As used herein, a “token,” is a smaller, individual grouping of text from a larger source of text. Tokens may be broken up by word, pair of words, sentence, or other delimitations. Tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as chains, for example for use as a Markov chain or Hidden Markov Model.

Still referring to FIG. 7, generating language model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language clement; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 7, processor 704 may determine one or more language elements in response 732 by identifying and/or detecting associations between one or more language elements (including phonemes or phonological elements, morphemes or morphological elements, syntax or syntactic elements, semantics or semantic elements, and pragmatic elements) extracted from at least response 732, including without limitation mathematical associations, between such words. Associations between language elements and relationships of such categories to other such term may include, without limitation, mathematical associations, including without limitation statistical correlations between any language element and any other language element and/or Language elements. Processor 704 may compare an input such as a sentence from response 732 with a list of keywords or a dictionary to identify language elements. For example, processor 704 may identify whitespace and punctuation in a sentence and extract elements comprising a string of letters, numbers or characters occurring adjacent to the whitespace and punctuation. Processor 704 may then compare each of these with a list of keywords or a dictionary. Based on the determined keywords or meanings associated with each of the strings, processor 704 may determine an association between one or more of the extracted strings and a medical status of a subject, such as an association between the words “chest” and “pain” and a particular category of medical conditions. Associations may take the form of statistical correlations and/or mathematical associations, which may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory.

Still referring to FIG. 7, processor 704 may be configured to determine one or more language elements in response 732 using machine learning. For example, processor 704 may generate the language processing model by any suitable method, including without limitation a natural language processing classification algorithm; language processing model may include a natural language process classification model that enumerates and/or derives statistical relationships between input terms and output terms. An algorithm to generate language processing model may include a stochastic gradient descent algorithm, which may include a method that iteratively optimizes an objective function, such as an objective function representing a statistical estimation of relationships between terms, including relationships between input language elements and output patterns or conversational styles in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted word, phrase, and/or other semantic unit. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Still referring to FIG. 7, processor 704 may be configured to determine one or more language elements in response 732 using machine learning by first creating or receiving language classification training data. Training data may include data containing correlations that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Still referring to FIG. 7, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below.

Still referring to FIG. 7, language classification training data may be a training data set containing associations between language element inputs and associated language element outputs. Language element inputs and outputs may be categorized by communication form such as written language elements, spoken language elements, typed language elements, or language elements communicated in any suitable manner. Language elements may be categorized by component type, such as phonemes or phonological elements, morphemes or morphological elements, syntax or syntactic elements, semantics or semantic elements, and pragmatic elements. Associations may be made between similar communication types of language elements (e.g. associating one written language element with another written language element) or different language elements (e.g. associating a spoken language element with a written representation of the same language element). Associations may be identified between similar communication types of two different language elements, for example written input consisting of the syntactic element “that” may be associated with written phonemes /th/, /ă/, and/t/. Associations may be identified between different communication forms of different language elements. For example, the spoken form of the syntactic element “that” and the associated written phonemes above. Language classification training data may be created using a classifier such as a language classifier. An exemplary classifier may be created, instantiated, and/or run using processor 704, or another computing device. Language classification training data may create associations between any type of language element in any format and other type of language element in any format. Additionally, or alternatively, language classification training data may associate language element input data to a medical condition. For example, language classification training data may associate occurrences of the syntactic elements “blurry” and “vision,” in a single sentence with a medical condition associated with vision and/or the eye.

Still referring to FIG. 7, processor 704 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A)P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 704 may then calculate a likelihood table by calculating probabilities of different data entries and classification labels. Processor 704 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

Still referring to FIG. 7, processor 704 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

Still referring to FIG. 7, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 7, language processing module may use a corpus of documents to generate associations between language elements in a language processing module, and a diagnostic engine may then use such associations to analyze words extracted from one or more documents and determine that the one or more documents indicate significance of a category. In an embodiment, a computing device may perform this analysis using a selected set of significant documents, such as documents identified by one or more experts as representing good information; experts may identify or enter such documents via graphical user interface, or may communicate identities of significant documents according to any other suitable method of electronic communication, or by providing such identity to other persons who may enter such identifications into a computing device. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York. In some embodiments, a transcript of a response produced using an automatic speech recognition system may be interpreted using a language model.

Still referring to FIG. 7, system 700 may determine medical condition category 736. As used herein, a “medical condition category” is a datum describing a particular subset of medical conditions. Non-limiting examples of medical condition categories include respiratory conditions and cardiac conditions. In some embodiments, system 700 may classify response 732 to medical condition category 736 using a medical condition classifier. Medical condition classifier may be trained using a supervised learning algorithm. Medical condition classifier may include a neural network. Medical condition classifier may be trained on a training dataset including example responses, associated with example medical condition categories. Such a training dataset may be obtained by, for example, obtaining deidentified historical medical records and correlating descriptions of symptoms by subjects with diagnoses received by such subjects. Once medical condition classifier is trained, it may be used to determine medical condition category 736. System 700 may input response 732 into medical condition classifier, and system 700 may receive medical condition category 736 from the model. In some embodiments, medical condition classifier may include a language model. In some embodiments, medical condition classifier may receive, as an input, an output of a language model and/or an automatic speech recognition system. In some embodiments, medical condition classifier may include a multimodal machine learning model. For example, medical condition classifier may accept as inputs, data describing a subject text and/or verbal description of a condition as well as an image of a body part of such subject. In some embodiments, medical condition classifier may accept as inputs embeddings of data, such as embeddings of text and/or image data. In some embodiments, system 700 may gather feedback on outputs of medical condition classifier. For example, feedback may be gathered by outputting, to subject and/or medical professional, medical condition category 736 and subject and/or medical professional may indicate a degree to which medical condition category 736 is accurate. In some embodiments, system 700 may augment and/or modify a training dataset used to train medical condition classifier based on such feedback. For example, system 700 may generate additional training data indicating that particular medical condition categorizations are incorrect. In some embodiments, medical condition classifier may be retrained on augmented and/or modified training data. In some embodiments, system 700 does not determine a medical condition category 736. In some embodiments, where response 732 indicates that no medical condition is present, system 700 may determine a medical condition category 736 associated with no medical condition.

Still referring to FIG. 7, system 700 may generate a medical condition report 740 as a function of medical condition category 736. As used herein, a “medical condition report” is a datum describing a document suitable for display to a human, where the document includes a medical condition category. System 700 may generate medical condition report 740 by including medical condition category 736 in a document format such as, in non-limiting examples, a Microsoft word document, a PDF document, or a text document. In some embodiments, medical condition report may include additional information, such as a measurement 744 of a subject. Measurement 744 may be detected and/or recorded by sensor 748 and may be transmitted to computing device 716. As used herein, a “measurement” of a subject is a quantitative or qualitative value associated with a specific parameter of a subject. In a non-limiting example, sensor 748 may include an optical sensor and measurement 744 may include and/or be derived from optical data. For example, measurement 744 may include an image of a body part of subject containing a symptom of a medical condition. In another non-limiting example, sensor 748 may include a scale and measurement 744 may include a weight reading taken by the scale. In some embodiments, system 700 may, using a remote sensor, take a measurement of a subject as a function of medical condition category 736. For example, system 700 may determine which of a plurality of potential measurements are relevant to a particular medical condition category 736 and determine which measurements to take based on relevance and available sensors at a location of a subject. In some embodiments, system 700 may receive a measurement of a subject from a wearable sensor and/or may generate medical condition report 740 as a function of such measurement. In a non-limiting example, subject may wear a device capable of detecting and/or recording medical information, such as a smartwatch, and system 700 may receive a current measurement and/or one or more historical measurements from such wearable sensor and/or a database associated with such wearable sensor. For example, system 700 may receive, from a wearable sensor and/or an associated database, measurements such as heart rate, measurements of physical activity, and measurements of time spent sleeping. Non-limiting examples of wearable devices and/or sensors include glucose monitors, motion trackers, and heart rate monitors. In some embodiments, a measurement detected by a wearable sensor may be used to generate and/or included within medical condition report 740. In some embodiments, which such measurements are included may be determined as a function of medical condition category, such as based on relevance of the measurement.

Still referring to FIG. 7, system 700 may generate medical condition report as a function of a historical measurement of a subject. For example, system 700 may receive from electronic health record database 756 a historical electronic health record 752 comprising a historical measurement of the subject and generate medical condition report 740 as a function of the historical measurement. In some embodiments, system 700 may generate medical condition report 740 as a function of a comparison between the historical measurement and the first measurement. For example, system 700 may determine a change, lack of change, trajectory, or the like based on one or more historical measurements and/or a current measurement. For example, system 700 may determine a degree to which a current weight of a subject is different from a historical weight of the subject, and may include such information, where relevant, in medical condition report 740.

Still referring to FIG. 7, system 700 may initiate a telemedicine session by connecting a subject with a medical professional as a function of response 732 and/or medical condition category 736 and transmitting medical condition report 740 to a device operated by the medical professional. For example, system 700 may identify a medical professional specializing in medical condition category 736 and may connect a subject to such medical professional. In another example, system 700 may determine whether to connect a subject with a medical professional as a function of content of response 732, such as based on whether response 732 indicates that a medical condition is present. As used herein, a “telemedicine session” is a channel supporting live communication between a subject and a medical professional. Such a channel may include, in non-limiting examples, a channel supporting audio data, a channel supporting video data, a channel supporting text data, or a combination thereof. In some embodiments, initiation of a telemedicine session may include connecting subject device 724 with medical professional device 764.

Still referring to FIG. 1, system 700 may initiate a telemedicine session using a communication channel interface. A communication channel interface may be used to connect subject device 724 with medical professional device 764. As used herein, a “communication channel interface” is a communication medium within an interface. A communication channel interface may include an application, script, and/or program capable of providing a means of communication between at least two parties, including any oral and/or written forms of communication. A communication channel interface may allow subject device 724 and/or medical professional device 764 to interface with electronic devices through graphical icons, audio indicators including primary notation, text based user interfaces, typed command labels, text navigation, and the like. A communication channel interface may include slides or other commands that may allow a user to select one or more options. A secure communication channel interface may include free form textual entries, where a user may type in a response and/or message. A secure communication channel interface may include a display interface. A display interface may include a form or other graphical element having display fields, where one or more elements of information may be displayed. Display interface may display data output fields including text, images, or the like containing one or more messages. A communication channel interface may include data input fields such as text entry windows, drop-down lists, buttons, checkboxes, radio buttons, sliders, links, or any other data input interface that may capture user interaction as may occur to persons skilled in the art upon reviewing the entirety of this disclosure. A communication channel interface may be provided, without limitation, using a web browser, a native application, a mobile application, and the like.

Still referring to FIG. 7, system 700 may generate an electronic health record 760 by recording audio data of the telemedicine session and generating a telemedicine session transcript by transcribing the audio data. In some embodiments, generating electronic health record 760 may include interpreting the telemedicine session transcript using a language model. Interpretation of a telemedicine session transcript may be used to, in non-limiting examples, identify a symptom described by a subject, identify a diagnosis made by a medical professional, identify which information discussed in a telemedicine session is medically relevant such that it can be isolated from other information, and/or summarize a telemedicine session. Generation of transcripts and interpretation of transcripts is described above. In some embodiments, electronic health record 760 may include measurement 744, medical condition category 736, response 732, prompt 720, and/or medical condition report 740. In some embodiments, computing device 716 may transmit electronic health record 760 to electronic health record database 756.

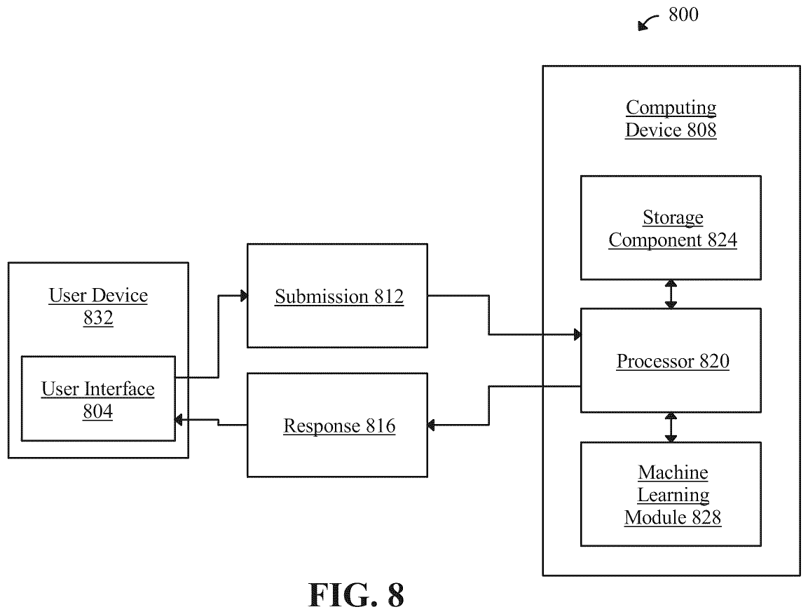

Now referring to FIG. 8, in some embodiments, apparatus 800 may communicate with user and/or instructor using a chatbot. According to some embodiments, user interface 804 on user device 832 may be communicative with a computing device 808 that is configured to operate a chatbot. In some embodiments, user interface 804 may be local to user device 832. In some embodiments, user interface 804 may be local to computing device 808. Alternatively, or additionally, in some cases, user interface 804 may remote to user device 832 and communicative with user device 832, by way of one or more networks, such as without limitation the internet. Alternatively, or additionally, one or more user interfaces may communicate with computing device 808 using telephonic devices and networks, such as without limitation fax machines, short message service (SMS), or multimedia message service (MMS). Commonly, user communicate with computing device 808 using text-based communication, for example without limitation using a character encoding protocol, such as American Standard for Information Interchange (ASCII). Typically, user interfaces conversationally interface with a chatbot, by way of at least a submission, from a user interface to the chatbot, and a response, from the chatbot to the user interface. For example, user interface 804 may interface with a chatbot using submission 812 and response 816. In some embodiments, submission 812 and/or response 816 may use text-based communication. In some embodiments, submission 812 and/or response 816 may use audio communication.

Still referring to FIG. 8, submission 812, once received by computing device 808 operating a chatbot, may be processed by a processor 820. In some embodiments, processor 820 processes submission 812 using one or more of keyword recognition, pattern matching, and natural language processing. In some embodiments, processor employs real-time learning with evolutionary algorithms. In some cases, processor 820 may retrieve a pre-prepared response from at least a storage component 824, based upon submission 812. Alternatively or additionally, in some embodiments, processor 820 communicates a response 816 without first receiving a submission, thereby initiating conversation. In some cases, processor 820 communicates an inquiry to user interface 804; and processor 820 is configured to process an answer to the inquiry in a following submission from the user interface. In some cases, an answer to an inquiry present within a submission from a user device may be used by computing device 808 as an input to another function. In some embodiments, computing device 808 may include machine learning module 828. Machine learning module 828 may include any machine learning models described herein. In some embodiments, submission 812 may be input into a trained machine learning model within machine learning module 828. In some embodiments, submission 812 may undergo one or more processing steps before being input into a machine learning model. In some embodiments, submission 812 may be used to train a machine learning model within machine learning module 828.

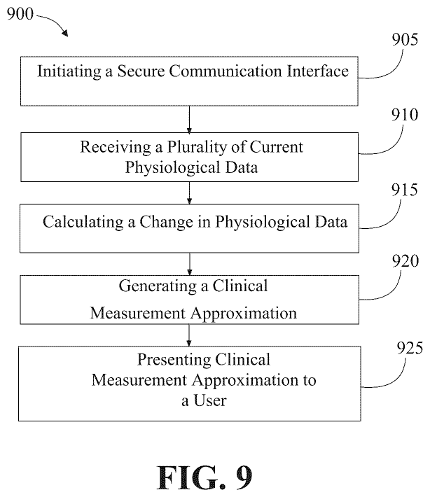

Referring now to FIG. 9, an exemplary embodiment of a method 900 of telemedicine diagnostics through remote sensing is illustrated. At step 905, a computing device 104 initiates a secure communication interface between the computing device and a client device 112 associated with a human subject 116 and at a second location, wherein the secure communication interface includes an audiovisual streaming protocol; this may be implemented, without limitation, as described above in reference to FIGS. 1-8.

At step 910, and with continued reference to FIG. 9, computing device 104 receives, from at least a remote sensor 204 at the second location, a plurality of current physiological data 208 associated with the human subject 116, wherein the plurality of current physiological data 208 comprises a first discrete set of current physiological data and a second discrete set of current physiological data; this may be implemented, without limitation, as described above in reference to FIGS. 1-8.

At step 915, and still referring to FIG. 9, computing device 104 calculates a change in physiological data between the first discrete set of current physiological data 208 and the second discrete set of current physiological data 208; this may be implemented, without limitation, as described above in reference to FIGS. 1-8.

Continuing in reference to FIG. 9, at step 920, computing device 104 generates a clinical measurement approximation 212 as a function of the change between the first discrete set and the second discrete set, wherein generating includes receiving approximation training data correlating physiological data with clinical measurement data, training a measurement approximation model as a function of the training data and a machine-learning process, and generating the clinical measurement approximation as a function of the change in physiological data and the trained measurement approximation model. Training the clinical measurement approximation model may include classification of the human subject to the approximation training data. Training the measurement approximation model may include generating a general model as a function of general training data and training a subject-specific model as a function of subject-specific training data. The first discrete set of current physiological data is temporally separated from the second discrete set of current physiological data; this may be implemented, without limitation, as described above in reference to FIGS. 1-8.

Continuing in reference to FIG. 9, continuing at step 920, computing device 104 may be further configured to record the first discrete set of current physiological data, generate a prompt instructing the human subject to perform an activity, and record the second discrete set of current physiological data. The computing device 104 may be further configured to verify that the human subject has performed the activity. The computing device 104 may be configured to determine a degree of reliability of the first clinical measurement and provide the degree of reliability using the communication interface. Computing device 104 may be further configured to identify a follow-up action as a function of the degree of reliability. System 100 may include receiving an instruction via the computing device 104.

Further referring to FIG. 9, generating clinical measurement approximation 212 may include calculating a change between a first discrete data set of the plurality of current physiological data 208 and a second discrete set of current physiological data 208 and generating the clinical measurement approximation 212 as a function of the change in physiological data and the trained measurement approximation model. First discrete set of current physiological data 208 may be temporally separated from second discrete set of current physiological data 208. In an embodiment, computing device 104 may record first discrete set of current physiological data 208, generate a prompt instructing human subject 116 to perform an activity, and record second discrete set of current physiological data 208. Computing device 104 may verify that human subject 116 has performed the activity.

At step 925, and still referring to FIG. 9, computing device 104 is configured to present, via the audiovisual streaming protocol of the secure communication interface, the clinical measurement approximation to a user of the computing device 104; this may be implemented, without limitation, as described above in reference to FIGS. 1-8.

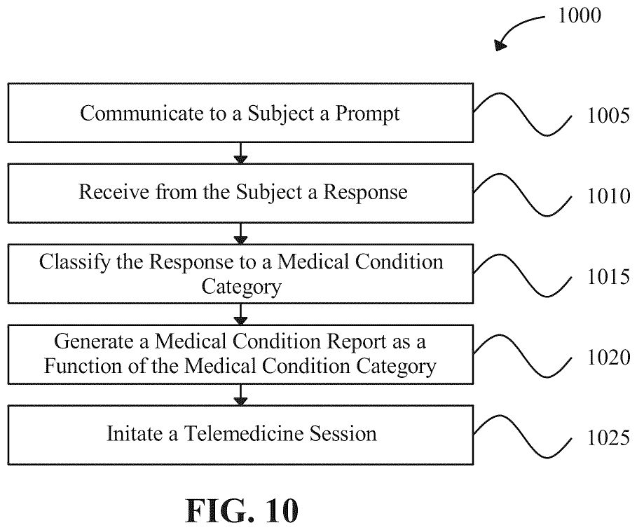

Referring now to FIG. 10, an exemplary embodiment of a method 1000 of telemedicine diagnostics through remote sensing is illustrated. One or more steps if method 1000 may be implemented, without limitation, as described with reference to other figures. One or more steps of method 1000 may be implemented, without limitation, using at least a processor.

Still referring to FIG. 10, in some embodiments, method 1000 may include a step 1005 of communicating to a subject a prompt

Still referring to FIG. 10, in some embodiments, method 1000 may include a step 1010 of receiving from the subject a response

Still referring to FIG. 10, in some embodiments, method 1000 may include a step 1015 of classifying the response to a medical condition category. In some embodiments, classifying the response to a medical condition category includes training a medical condition category machine learning model on a training dataset including a plurality of example responses correlated to a plurality of example medical condition categories; and generating the medical condition category as a function of the response using the trained medical condition category machine learning model.

Still referring to FIG. 10, in some embodiments, method 1000 may include a step 1020 of generating a medical condition report as a function of the medical condition category.

Still referring to FIG. 10, in some embodiments, method 1000 may include a step 1025 of initiating a telemedicine session. In some embodiments, step 1025 may include connecting the subject with a medical professional as a function of the response, and/or transmitting the medical condition report to a device operated by the medical professional.

Still referring to FIG. 10, in some embodiments, method 1000 may further include using a remote sensor, taking a first measurement of the subject as a function of the medical condition category; and generating the medical condition report as a function of the first measurement.

Still referring to FIG. 10, in some embodiments, method 1000 may further include receiving from an electronic health record database a historical electronic health record comprising a historical measurement of the subject; and generating the medical condition report as a function of the historical measurement.

Still referring to FIG. 10, in some embodiments, method 1000 may further include receiving, from a wearable device, a second measurement of the subject; and generating the medical condition report as a function of the second measurement. In some embodiments, the wearable device comprises a glucose monitor. In some embodiments, the wearable device comprises a motion tracker. In some embodiments, the wearable device comprises a heart rate monitor.

Still referring to FIG. 10, in some embodiments, method 1000 may further include generating an electronic health record by recording audio data of the telemedicine session; and generating a telemedicine session transcript by transcribing the audio data. In some embodiments, generating the electronic health record further comprises interpreting the telemedicine session transcript using a language model.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

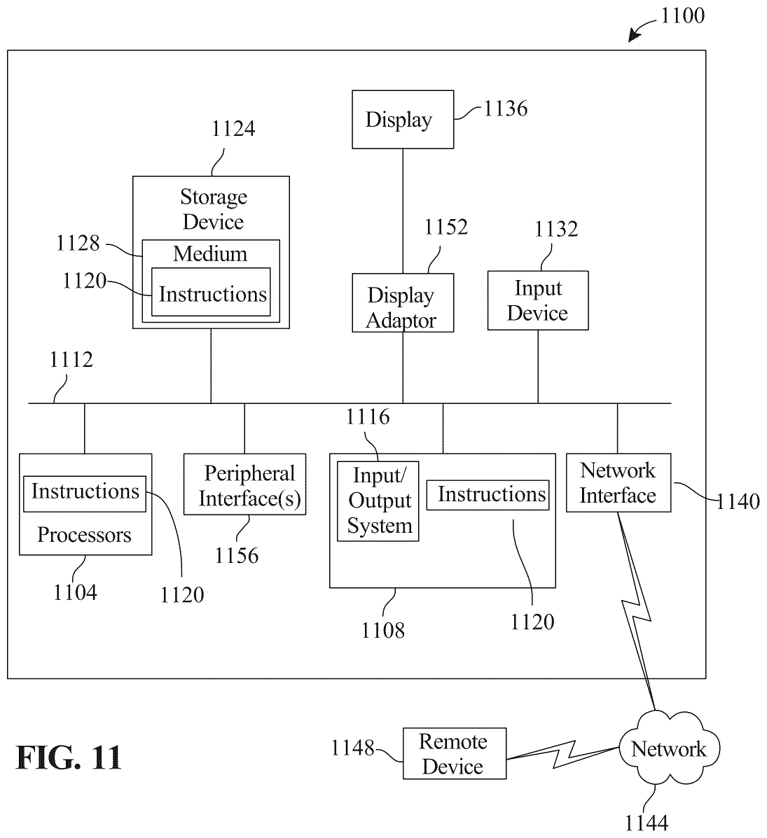

FIG. 11 shows a diagrammatic representation of one embodiment of a computing device in the exemplary form of a computer system 1100 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1100 includes a processor 1104 and a memory 1108 that communicate with each other, and with other components, via a bus 1112. Bus 1112 may include any of several types of bus structures including, but not limited to, a memory bus, a memory controller, a peripheral bus, a local bus, and any combinations thereof, using any of a variety of bus architectures.

Processor 1104 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1104 may be organized according to Von Neumann and/or Harvard architecture as a non-limiting example. Processor 1104 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1108 may include various components (e.g., machine-readable media) including, but not limited to, a random-access memory component, a read only component, and any combinations thereof. In one example, a basic input/output system 1116 (BIOS), including basic routines that help to transfer information between elements within computer system 1100, such as during start-up, may be stored in memory 1108. Memory 1108 may also include (e.g., stored on one or more machine-readable media) instructions (e.g., software) 1120 embodying any one or more of the aspects and/or methodologies of the present disclosure. In another example, memory 1108 may further include any number of program modules including, but not limited to, an operating system, one or more application programs, other program modules, program data, and any combinations thereof.

Computer system 1100 may also include a storage device 1124. Examples of a storage device (e.g., storage device 1124) include, but are not limited to, a hard disk drive, a magnetic disk drive, an optical disc drive in combination with an optical medium, a solid-state memory device, and any combinations thereof. Storage device 1124 may be connected to bus 1112 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1124 (or one or more components thereof) may be removably interfaced with computer system 1100 (e.g., via an external port connector (not shown)). Particularly, storage device 1124 and an associated machine-readable medium 1128 may provide nonvolatile and/or volatile storage of machine-readable instructions, data structures, program modules, and/or other data for computer system 1100. In one example, software 1120 may reside, completely or partially, within machine-readable medium 1128. In another example, software 1120 may reside, completely or partially, within processor 1104.

Computer system 1100 may also include an input device 1132. In one example, a user of computer system 1100 may enter commands and/or other information into computer system 1100 via input device 1132. Examples of an input device 1132 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1132 may be interfaced to bus 1112 via any of a variety of interfaces (not shown) including, but not limited to, a serial interface, a parallel interface, a game port, a USB interface, a FIREWIRE interface, a direct interface to bus 1112, and any combinations thereof. Input device 1132 may include a touch screen interface that may be a part of or separate from display device 1136, discussed further below. Input device 1132 may be utilized as a user selection device for selecting one or more graphical representations in a graphical interface as described above.

A user may also input commands and/or other information to computer system 1100 via storage device 1124 (e.g., a removable disk drive, a flash drive, etc.) and/or network interface device 1140. A network interface device, such as network interface device 1140, may be utilized for connecting computer system 1100 to one or more of a variety of networks, such as network 1144, and one or more remote devices 1148 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1144, may employ a wired and/or a wireless mode of communication. In general, any network topology may be used. Information (e.g., data, software 1120, etc.) may be communicated to and/or from computer system 1100 via network interface device 1140.

Computer system 1100 may further include a video display adapter 1152 for communicating a displayable image to a display device, such as display device 1136. Examples of a display device include, but are not limited to, a liquid crystal display (LCD), a cathode ray tube (CRT), a plasma display, a light emitting diode (LED) display, and any combinations thereof. Display adapter 1152 and display device 1136 may be utilized in combination with processor 1104 to provide graphical representations of aspects of the present disclosure. In addition to a display device, computer system 1100 may include one or more other peripheral output devices including, but not limited to, an audio speaker, a printer, and any combinations thereof. Such peripheral output devices may be connected to bus 1112 via a peripheral interface 1156. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for telemedicine diagnostics through remote sensing, the system comprising:
at least a processor; and
a memory communicatively connected to the at least processor, wherein the memory contains instructions configuring the at least processor to:
communicate to a subject a prompt;
receive from the subject a response;
classify the response to a medical condition category;
generate a medical condition report as a function of the medical condition category; and
initiate a telemedicine session by:
using a communication channel interface, connecting the subject with a medical professional as a function of the response; and
transmitting the medical condition report to a device operated by the medical professional.

2. The system of claim 1, wherein the memory contains instructions configuring the at least processor to:
using a remote sensor, take a first measurement of the subject as a function of the medical condition category; and
generate the medical condition report as a function of the first measurement.

3. The system of claim 2, wherein the memory contains instructions configuring the at least processor to:
receive from an electronic health record database a historical electronic health record comprising a historical measurement of the subject; and
generate the medical condition report as a function of a comparison between the historical measurement and the first measurement.

4. The system of claim 1, wherein the memory contains instructions configuring the at least processor to:
receive, from a wearable device, a second measurement of the subject; and
generate the medical condition report as a function of the second measurement.

5. The system of claim 4, wherein the wearable device comprises a glucose monitor.

6. The system of claim 4, wherein the wearable device comprises a motion tracker.

7. The system of claim 4, wherein the wearable device comprises a heart rate monitor.

8. The system of claim 1, wherein classifying the response to a medical condition category comprises:
training a medical condition category machine learning model on a training dataset including a plurality of example responses correlated to a plurality of example medical condition categories; and
generating the medical condition category as a function of the response using the trained medical condition category machine learning model.

9. The system of claim 1, wherein the memory contains instructions configuring the at least processor to generate an electronic health record by:
recording audio data of the telemedicine session; and
generating a telemedicine session transcript by transcribing the audio data.

10. The system of claim 9, wherein generating the electronic health record further comprises interpreting the telemedicine session transcript using a language model.

11. A method of telemedicine diagnostics through remote sensing, the method comprising:
using at least a processor, communicating to a subject a prompt;
using the at least a processor, receiving from the subject a response;
using the at least a processor, classifying the response to a medical condition category;
using the at least a processor, generating a medical condition report as a function of the medical condition category; and
using the at least a processor, initiating a telemedicine session by:
using a communication channel interface, connecting the subject with a medical professional as a function of the response; and
transmitting the medical condition report to a device operated by the medical professional.

12. The method of claim 11, wherein the method further comprises:
using a remote sensor, taking a first measurement of the subject as a function of the medical condition category; and
generating the medical condition report as a function of the first measurement.

13. The method of claim 12, wherein the method further comprises:
receiving from an electronic health record database a historical electronic health record comprising a historical measurement of the subject; and
generating the medical condition report as a function of as a function of a comparison between the historical measurement and the first measurement.

14. The method of claim 11, wherein the method further comprises:
receiving, from a wearable device, a second measurement of the subject; and
generating the medical condition report as a function of the second measurement.

15. The method of claim 14, wherein the wearable device comprises a glucose monitor.

16. The method of claim 14, wherein the wearable device comprises a motion tracker.

17. The method of claim 14, wherein the wearable device comprises a heart rate monitor.

18. The method of claim 11, wherein classifying the response to a medical condition category comprises:
training a medical condition category machine learning model on a training dataset including a plurality of example responses correlated to a plurality of example medical condition categories; and
generating the medical condition category as a function of the response using the trained medical condition category machine learning model.

19. The method of claim 11, wherein generating an electronic health record further comprises:
recording audio data of the telemedicine session; and
generating a telemedicine session transcript by transcribing the audio data.

20. The method of claim 19, wherein generating the electronic health record further comprises interpreting the telemedicine session transcript using a language model.
